SEQUENCE LISTING (17 sequences)

SEQ ID NO: 1 - protein, 275 aa, synthetic construct
AQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGAYNGTCMASAHVAGAAALILSKHPNWTNTQVRSSLENTTTKLGDSFYYGKGLINVQAAAQ

SEQ ID NO: 2 - protein, 275 aa, synthetic construct
AQSVPYGVSQIKAPALHSQGYTGSNVKVAVIDSGIDSSHPDLKVAGGASFVPSETNPFQDNNSHGTHVAGTVAALDNSIGVLGVAPSASLYAVKVLGADGSGQYSWIISGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNRYGAYSGTCMASAHVAGAAALILSKHPNWTNTQVRSSLENTTTKLGDSFYYGKGLINVQAAAQ

SEQ ID NO: 3 - protein, 275 aa, synthetic construct
AQSVPYGVSQIKAPALHSQGYTGSNVKVAVIDSGIDSSHPDLKVAGGASFVPSETNPFQDNNSHGTHVAGTVAALDNSIGVLGVAPSASLYAVKVLGADGSGQYSWIISGIEWAIANNMDVINLALGGPSGSAALKAAVDKAVASGVVVVAAAGNEGTSGSSSTVGYPGKYPSVIAVGAVDSSNQRASFSSVGPELDVMAPGVSIQSTLPGNRYGAYSGTCMASAHVAGAAALILSKHPNWTNTQVRSSLENTTTKLGDSFYYGKGLINVQAAAQ

SEQ ID NO: 4 - protein, 266 aa, synthetic construct
AQSVPYGVSQIKAPALHSQGYTGSNVKVAVIDSGIDSSHPDLNVAGGASFVPSETNPFQDNNSHGTHVAGTVLAVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIVSTLPGNKYGAKSGTCMASAHVAGAAALILSKHPNWTNTQVRSSLENTTTKLGDSFYYGKGLINVEAAAQ

SEQ ID NO: 5 - protein, 8 aa, synthetic construct
ENLYFQSY

SEQ ID NO: 6 - protein, 8 aa, synthetic construct
ENLYFQSK

SEQ ID NO: 7 - protein, 8 aa, synthetic construct
ENLYFQSA

SEQ ID NO: 8 - protein, 4 aa, synthetic construct
AAPY

SEQ ID NO: 9 - protein, 4 aa, synthetic construct
AAPK

SEQ ID NO: 10 - protein, 4 aa, synthetic construct
AAPA

SEQ ID NO: 11 - protein, 7 aa, synthetic construct
  VARIANT 7: note = Wherein the S at position 7 may also be G or A
EXXYXQS

SEQ ID NO: 12 - protein, 7 aa, synthetic construct
  VARIANT 2: note = Wherein the X at position 2 may be T or V
EXLFQGP

SEQ ID NO: 13 - protein, 5 aa, synthetic construct
DDDDK

SEQ ID NO: 14 - protein, 4 aa, synthetic construct
  VARIANT 2: note = Wherein the D at position 2 may also be E
IDGR

SEQ ID NO: 15 - protein, 4 aa, synthetic construct
LVPR

SEQ ID NO: 16 - skipped

SEQ ID NO: 17 - protein, 4 aa, synthetic construct
IEPD

(19) United States
(12) Patent Application Publication
(10) Pub. No.: US 2025/0189531 A1
(43) Pub. Date: Jun. 12, 2025

(54) Glycan-Tethered Stabiligases

(71) Applicant: The Regents of the University of California, Oakland, CA (US)

(72) Inventors: James A. Wells, San Francisco, CA (US); Kaitlin Schaefer, San Francisco, CA (US)

(21) Appl. No.: 18/905,583

(22) Filed: Oct. 3, 2024

Related U.S. Application Data
(63) Continuation of application No. PCT/US2023/065379, filed on Apr. 5, 2023.
(60) Provisional application No. 63/327,767, filed on Apr. 5, 2022.

(51) Int. Cl. G01N 33/574; G01N 33/548; G01N 33/68

(52) U.S. Cl. CPC G01N 33/57492; G01N 33/548; G01N 33/6842

(57) ABSTRACT

Provided herein are novel stabiligases for use in cell membrane proteome analysis. The subject stabiligases are capable of attaching to glycans found on the surface of cell membranes to form glycan-tethered (GT) stabiligases. Such glycan-tethered stabiligases are capable of robustly and selectively attaching label probes to cell surface proteins of intact cells. The subject novel stabiligases described herein advantageously allow for the identification and profiling of cell membrane proteins that have undergone an extracellular N-terminal proteolytic event.

### CROSS REFERENCE TO RELATED APPLICATIONS

The present disclosure claims priority to U.S. Provisional Patent Application No. 63/327,767, filed on Apr. 5, 2022, which is hereby incorporated by reference in its entirety.

### GOVERNMENT SUPPORT

This invention was made with government support under grant R01 CA248323 awarded by The National Institutes of Health. The government has certain rights in the invention.

### INCORPORATION BY REFERENCE OF SEQUENCE LISTING PROVIDED AS A SEQUENCE LISTING XML FILE

A Sequence Listing is provided herewith as a Sequence Listing XML, UCSF-763CON_SEQ_LIST, created on Dec. 20, 2024 and having a size of 17,330 bytes. The contents of the Sequence Listing XML are incorporated herein by reference in their entirety.

### BACKGROUND

The cell surface proteome comprises approximately 3,000 proteins and is functionally critical for cellular fate and response to environmental stimuli¹. Whereas intracellular proteins may be functionally altered by hundreds of chemically different post-translational modifications (PTMs)², proteins in the extracellular space are far more limited in variety. Proteolysis is distinctly prominent among cell surface PTMs, and membrane-embedded and secreted proteases modulate many processes including cell-cell interactions, signal transduction, and cytokine secretion³. Consequently, dysregulated proteolysis is associated with many diseases. Cleavage events on healthy and abnormal cells alike create proteoforms that commonly display extracellular neo-N-termini (FIG. 1A). Characterizing cleavage events may reveal new proteoforms that can be selectively targeted for immunotherapy⁴.

Global identification of proteolysis has been greatly improved by advances in mass spectrometry (MS) methods but characterizing extracellular proteolytic modifications are challenging subjects with current techniques⁵⁻⁸. One common approach is to isolate proteins that are proteolytically-shed into the supernatant of cell cultures⁹. Despite generating data on hundreds of shedding events, this method does not precisely identify cleavage sites and is primarily limited to proteins cleaved close to or within the membrane. Another approach is to identify proteolytic products via C- or N-terminal labeling in cell lysates^{7,10-12}. Labeling takes place within the whole cell lysate and the high complexity of the proteome, as well as the challenging properties of many membrane proteins-most frequently poor stability and low abundance relative to intracellular proteins-leads to incomplete coverage of extracellular proteolysis. Thus, there remains a need for improved compositions and methods for analysis of the cell surface proteome.

### SUMMARY

Provided herein are novel stabiligases for use in cell membrane proteome analysis. The subject stabiligases are capable of attaching to glycans found on the surface of cell membranes to form glycan-tethered (GT) stabiligases. Such glycan-tethered stabiligases are capable of robustly and selectively attaching label probes to cell surface proteins, particular those that have undergone an extracellular proteolytic event. The subject novel stabiligases described herein advantageously allow for the identification and profiling of cell membrane proteins that have undergone an extracellular N-terminal proteolytic event. The subject stabiligases and methods provided can be used to study how proteases remodel the extracellular proteome across healthy and malignant cells. Moreover, identification of neo-epitopes using the subject compositions and methods provided herein can be used for therapeutic targeting.

In one aspect, provided herein is a cell comprising a stabiligase tethered to extracellular glycans on intact human cells. In some embodiments, the stabiligase is tethered to a glycan on the cell membrane protein by an oxime or a hydrazone bond. In certain embodiments, the cell is a mammalian cell. In exemplary embodiments, the cell is a human cell. In some embodiments, the cell is a cancer cell.

Also provided herein are methods for making a cell comprising a glycan-tethered stabiligase, comprising oxidative coupling a stabiligase to a cell-membrane protein on the surface of the cell. In some embodiments, the method comprises: a) providing a cell comprising a cell membrane protein with an aldehyde group; b) contacting the cell with a stabiligase comprising a nucleophilic group under conditions wherein the aldehyde group of the cell membrane protein and the nucleophilic group form a bond, thereby tethering the stabiligase to the cell membrane protein of the cell. In some embodiments, the cell membrane protein comprises a glycan comprising the aldehyde group. In some embodiments, the nucleophilic group is an α-aminooxy- or α-hydrazido-group.

In another aspect, provided herein is a stabiligase comprising an N-terminal an α-aminooxy- or α-hydrazido-group. In some embodiments, the stabiligase is produced from a parental stabiligase having the amino acid sequence of any one of SEQ ID NOs: 1-4. In some embodiments, the stabiligase is a variant of a stabiligase having the amino acid sequence of any one of SEQ ID NOs: 1-4. In some embodiments, the stabiligase comprises at least 1, 2, 3, 4, 5, 6, 7, 8, 9, 10, 11, 12, 13, 14, 15, 16, 17, 18, 19 or 20 amino acid modifications as compared to the stabiligase of SEQ ID NO:1-4.

In another aspect, provided herein is a method of identifying a population of cell membrane proteins on a cell, the method comprising: a) providing a cell comprising a membrane-tethered stabiligase and a population of cell membrane proteins; b) contacting the cell with a label probe comprising an ester substrate and a detectable label under conditions wherein the membrane-tethered stabiligase attaches the label probe to the population of cell membrane proteins to form a population labelled cell membrane proteins; c) isolating the labelled cell membrane proteins; and d) analyzing the isolated labelled cell membrane proteins; therein identifying the population of cell membrane proteins on the cell. In some embodiments, the label probe comprises a capture ligand and the isolating step c) comprises capturing the labelled cell membrane proteins using a substrate comprising a capture moiety. In some embodiments, the capture ligand is biotin and the capture moiety selected from the group consisting of: avidin, streptavidin, neutravidin, and captavidin. In some embodiments, the detectable label is detectable by mass spectrometry. In some embodiments, the detectable label is an aminobutyric acid (Abu) tag. In exemplary embodiments, the cell membrane proteins are analyzed in step d) using liquid chromatography with tandem mass spectrometry (LC-MS/MS). In some embodiments, the cell is a cancer cell.

### BRIEF DESCRIPTION OF THE DRAWINGS

FIGS. 1A-IC depict an N-terminomics approach for capturing proteolytic neo-N-termini by chemically-tethering stabiligase to glycans on living cells. a) Proteases modify cell surface proteins and create new N-termini (neo-N-termini) often exposed within the extracellular environment. To characterize proteolytic modifications on the cell surface, we considered attaching the engineered ligase, stabiligase, to cell surfaces. In the presence of accessible N-termini, stabiligase tags α-amines with a peptide ester containing a biotin (blue), a TEV-protease cleavage site, and an amino-butyric acid mass tag (A, green sphere). After a LC-MS workflow, labeled N-termini peptides are identified using LC-MS-MS. b) Strategy for cell surface tethering of α-nucleophile stabiligase. Treating cells with a mild periodate condition creates aldehydes on extracellular glycans, which will then react with the N-terminal nucleophile on stabiligase. c) Synthetic scheme to conjugate an α-nucleophile onto the N-terminus of stabiligase. Purified stabiligase (A1S) is treated with Ellman's reagent to create a C221-adduct, and then incubated briefly with sodium periodate to generate an N-terminal aldehyde. The aldehyde then reacts completely with either excess bis-amino-oxy or bis-hydrazide-based reagents, and after a TNB-deprotection, functionalized α-nucleophilic-stabiligases are obtained.

FIGS. 2A-2F provide a summary of studies showing the attachment of glycan-tethered (GT) stabiligase to intact cell surfaces and N-termini labeling of membrane proteins. a) Treatment of HEK293T cells with sodium periodate and then GT-stabiligase shows significant enzyme tethering by flow cytometric analysis monitoring the C-terminal histidine tag on stabiligases with AF647 anti-His antibody staining. N-terminal aminooxy-group facilitated stabiligase tethering more efficiently than a N-terminal hydrazide. b) Fluorescence microscopy of HEK293T cells tethered to C-terminal, histidine-tagged GT-stabiligase shows exclusive membrane staining. c) Pre-treatment of HEK293T cells with Vibrio cholerae sialidase shows greatly reduced attachment of GT-stabiligase. d) Attachment of GT-stabiligase to cells significantly enhances labeling with the biotinylated peptide ester compared to soluble stabiligase as analyzed by flow cytometric analysis with AF488-streptavidin staining. e) Immunoblot detection of biotinylated proteins within the subcellular membrane fraction is consistent with flow cytometry staining of cells in D. f) Similar to the previous panel, the activity of different GT-stabiligases with longer linker lengths was evaluated using immunoblot detection of the membrane fraction. Lengthening the linker between the N-terminal aminooxy-group and the stabiligase domain does not affect ligation. Data are representative of at least three independent experiments with similar results. In a) and d), data is presented as the mean±s.e.m and the P values were calculated using a two-tailed unpaired Student's t-test.

FIGS. 3A-3E provide a summary of a studying, showing that surface N-terminomics with GT-stabiligase is a proteomic method for characterizing proteolytic neo-N-termini across cell types. a) Initial N-terminomics with GT-stabiligase was performed on HEK293T cells, which yielded 507 N-termini on 186 cell surface proteins. The distribution of membrane proteins mapped with N-termini was similar to the population ratios of different membrane proteins. N-termini peptides were grouped based on the location of cleavage: initiator methionine (Met), signal peptide, propeptide junction, transmembrane, and extracellular regions of proteins. The vast majority of cleavages (74%) mapped to extracellular regions of proteins and were localized either to linker regions or within domains that were predominantly predicted as beta-strands (data not shown). The icelogo of the P4-P4′ residues flanking the cut-site (scissors) shows a range of different residues at the P1 position. b) GT-stabiligase cell surface N-terminomics captures neo-N-termini across adherent cell types and primary immune cells. c) N-termini in panel b were compared to N-terminomics compiled in Topfind 4.1 and showed modest overlap. d) GO analysis of proteins with propeptide cleavages show significant enrichment in endopeptidases. These mature proteases represent different hydrolase families. e) Distance from the proteolytic event to the membrane was approximated by amino acid distances between the membrane anchor (transmembrane helix or GPI-anchor) and the cleavage site.

FIGS. 4A-4E provides a summary of studies, showing that oncogenes drive common and unique proteolytic cleavages on cell surfaces. a) Schematic depicting the application of quantitative N-terminomics with GT-stabiligase to identify differences in proteolytic, neo-N-termini in the presence of single oncogenes, her2 or kras(g12V). After growing in SILAC media, non-tumorigenic MCF10A cells were combined with cells harboring the single oncogenes for the GT-stabiligase workflow as described previously. b) Venn diagram for neo-N-termini for KRas^{G12V} or Her2 transformed cells that were substantially changed (1.8-fold threshold) in comparison to the control MCF10A cells. c) Protein classes represented by enriched neo-N-termini observed in the presence of either oncogene shows an enrichment of transmembrane signal receptors and cell adhesion proteins. d) A heat map represents the comparisons between overlapping N-termini in either the presence of Her2 or KRas^{G12V} and the protein abundances observed using CSC proteomics³⁸. There is only a weak correlation between changes in the neo-N-termini and expression levels of the proteins. e). Immunoblot analysis of select proteins (NOTCH2, DSG2, LDLR, CAD13) is consistent with quantitative proteolytic differences observed using GT-stabiligase N-terminomics. Independent experiments were performed in triplicate with similar results.

### DETAILED DESCRIPTION

### I. Overview

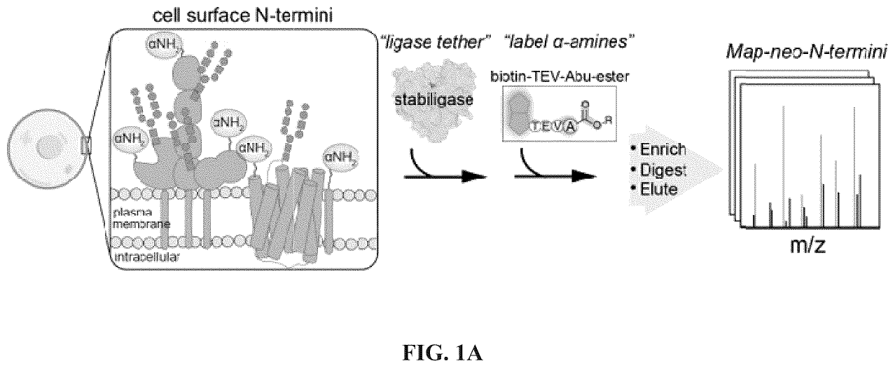

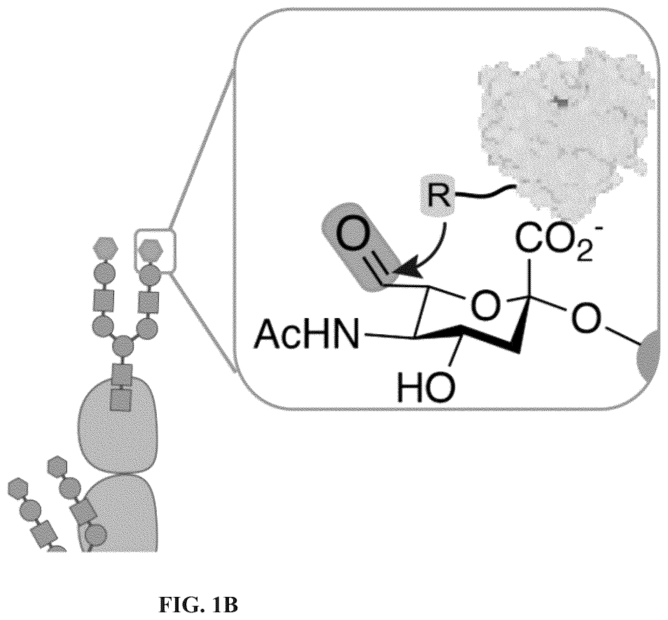

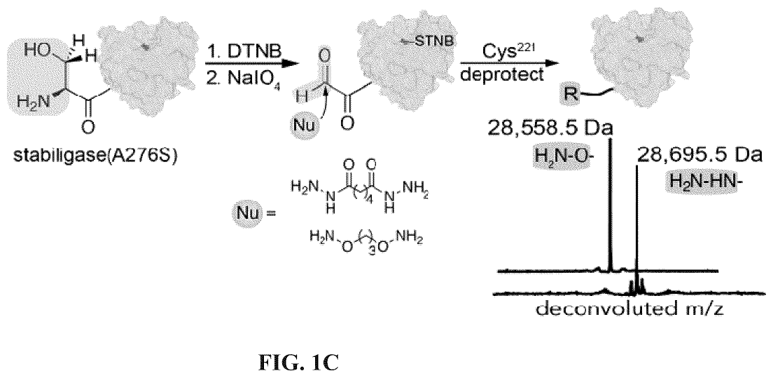

Previously, a proteomics technology (N-terminomics) was developed based on subtiligase, a mechanistically-engineered ligase that can specifically label N-terminal, α-amines on diverse proteins in a complex milieu (FIG. 1B)^{13,14}. The ligase, and a compilation of engineered variants, are used for diverse biotechnological applications, including peptide cyclization and protein synthesis¹⁵. Labeling proteolytic neo-N-termini in whole cell lysates with subtiligase, has allowed for the extensive annotation of substrates of soluble proteases like caspases¹⁷. In attempts to identify extracellular proteolysis, however, it was found that using soluble subtiligase to label intact cells or whole cell lysates yielded few cell surface N-termini.

Here, an N-terminomics approach was developed for characterizing extracellular proteolytic modifications across diverse cell types by chemical tethering of the subtiligase variant (stabiligase) to living cells. Aspects of the subject ligase and related methods are further detailed below

### II. Glycan-Tethered (GT) Stabiligases

In one aspect, provided herein are stabiligases that are capable of tethering to extracellular glycans on intact cells. Subject stabiligases that are tethered to the surface of intact cells are capable of labelling cell membrane proteins that have undergone an N-terminal extracellular proteolytic event. In particular, the membrane tethered stabiligases provided herein are capable of attaching label probes that comprises a peptide ester substrate to N-terminal α-amines of cell membrane proteins that have undergone a proteolytic event, thereby labelled cell membrane proteins. In some embodiments, the labelled cell membrane proteins are subsequently isolated and analyzed.

The subject stabiligases provided herein are modified to include an N-terminus α-nucleophilic moiety that allows the attachment of the subject ligase to an aldehyde group on a glycan of a cell membrane protein. In some embodiments, the N-terminus α-nucleophile is an α-aminooxy- or α-hydrazido-group. In embodiments of the subject stabiligase that includes an α-aminooxy-group, the α-aminooxy-group is capable of interacting with an aldehyde group of a glycan on a cell surface protein to form an oxime bond, thereby allowing the subject stabiligase to attach the surface of an intact cell. In some embodiments of the subject stabiligase that includes a α-hydrazido-group, the α-hydrazido-group is capable of interacting with an aldehyde group of a glycan on a cell surface protein to form a hydrazone bond.

To make the subject stabiligase, a parental stabiligase (e.g., a wildtype stabiligase) is subjected to auto-prodomain removal to generate an N-terminal alanine (A1). The N-terminal alanine is then mutated to serine (A1S) to create a vicinal α-amino alcohol. The N-terminal amino-alcohol is subsequently converted to a glyoxyl-aldehyde by sodium periodate oxidation. The ligase is then oxidized with periodate to generate an N-terminal aldehyde and then incubated with either bis-aminooxy- or bis-hydrazido-reagent. After a TNB deprotection, the subject N-terminus α-nucleophilic group is obtained. Suitable parental stabiligases that can be modified to make the subject ligases include those described in Chang et al., Proc. Natl. Acad. Sci. USA 91:12544-12548 (1994); Atwell et al., Proc. Natl. Acad. Sci. USA 96:9497-9502 (1999); and Weeks et al., Nat Chem Biol 14:50-57 (2018), and US20190185836A1 (see, e.g., SEQ ID NOs: 1-4) or biologically active variants thereof, which are incorporated by reference in their entirety and particularly in pertinent parts relating to stabiligases. In some embodiments, the parental stabiligase is one of the stabiligases in Table 1. In certain embodiments, the parental stabiligase is a biologically active variant of a stabiligase in Table 1. In exemplary embodiments, the parental stabiligase is a variant of a stabiligase in Table 1 that includes 1, 2, 3,4,5,6,7, 8, 9, 10, 11, 12, 13, 14, 15, 16, 17, 18, 19, or 20 amino acid modifications (e.g., an amino acid substitution) as compared to a stabiligase in Table 1. In some embodiments, the parental stabiligase is a biologically active stabiligase that is at least about 85%, 86%, 87%, 88%, 89%, 90%, 91%, 92%, 93%, 94%, 95%, 96%, 97%, 98%, or 99% identical to a stabiligase in Table 1.

TABLE 1
Stabiligase

AQSVPYGVSQIKAPALHSQGYTGSNVKVAVIDSGIDSSHPDLKVA
GGASMVPSETNPFQDNNSHGTHVAGTVAALNNSIGVLGVAPSASL
YAVKVLGADGSGQYSWIINGIEWAIANNMDVINMSLGGPSGSAAL
KAAVDKAVASGVVVVAAAGNEGTSGSSSTVGYPGKYPSVIAVGAV
DSSNQRASFSSVGPELDVMAPGVSIQSTLPGNKYGAYNGTCMASA
HVAGAAALILSKHPNWTNTQVRSSLENTTTKLGDSFYYGKGLINV
QAAAQ
(SEQ ID NO: 1)
AQSVPYGVSQIKAPALHSQGYTGSNVKVAVIDSGIDSSHPDLKVA
GGASFVPSETNPFQDNNSHGTHVAGTVAALDNSIGVLGVAPSASL
YAVKVLGADGSGQYSWIISGIEWAIANNMDVINMSLGGPSGSAAL
KAAVDKAVASGVVVVAAAGNEGTSGSSSTVGYPGKYPSVIAVGAV
DSSNQRASFSSVGPELDVMAPGVSIQSTLPGNRYGAYSGTCMASA
HVAGAAALILSKHPNWTNTQVRSSLENTTTKLGDSFYYGKGLINV
QAAAQ
(SEQ ID NO: 2)
AQSVPYGVSQIKAPALHSQGYTGSNVKVAVIDSGIDSSHPDLKVA
GGASFVPSETNPFQDNNSHGTHVAGTVAALDNSIGVLGVAPSASL
YAVKVLGADGSGQYSWIISGIEWAIANNMDVINLALGGPSGSAAL
KAAVDKAVASGVVVVAAAGNEGTSGSSSTVGYPGKYPSVIAVGAV
DSSNQRASFSSVGPELDVMAPGVSIQSTLPGNRYGAYSGTCMASA
HVAGAAALILSKHPNWTNTQVRSSLENTTTKLGDSFYYGKGLINV
QAAAQ
(SEQ ID NO: 3)
AQSVPYGVSQIKAPALHSQGYTGSNVKVAVIDSGIDSSHPDLNVA
GGASFVPSETNPFQDNNSHGTHVAGTVLAVAPSASLYAVKVLGAD
GSGQYSWIINGIEWAIANNMDVINMSLGGPSGSAALKAAVDKAVA
SGVVVVAAAGNEGTSGSSSTVGYPGKYPSVIAVGAVDSSNQRASF
SSVGPELDVMAPGVSIVSTLPGNKYGAKSGTCMASAHVAGAAALI
LSKHPNWTNTQVRSSLENTTTKLGDSFYYGKGLINVEAAAQ
(SEQ ID NO: 4)

### III. Cell Compositions

In another aspect, provided herein is a cell comprising a stabiligase tethered to an extracellular glycan. In some embodiments, a subject stabiligase that includes a N-terminus α-nucleophilic group forms a bond with an aldehyde on a glycan of a cell membrane protein, thereby tethering the subject ligase to the cell membrane surface. In some embodiments, cells of interest are treated with sodium periodate to form glycans with aldehydes for reacting with the subject ligases. The ligases are then contacted with the cell in the presence of an amine catalyst (e.g., aniline), that allows for bond formation (e.g., an oxime or hydrazone bond) between the ligase and cell.

The subject ligases provided herein can be attached to any suitable cell where cell surface proteome analysis is desired. In some embodiments, the cell is a target for drug development. In some embodiments, the cell is a cell that has been contacted with a particular therapy. In certain embodiments, the cell is a cell that is resistant to a particular therapy. In some embodiments, the cell is a mammalian cell, such as a mouse, rat, hamster, guinea pig, rabbit, sheep, goat, pig, monkey, human cell, and the like. In certain embodiments, the cell is a human cell. In exemplary embodiments, the cell is a cancer cell (e.g., a tumor cell, a circulating tumor cell, a bone marrow cell, a tissue biopsy). In certain embodiments, the cancer is a carcinoma, blastoma, and sarcoma, and certain leukemia or lymphoid malignancies. More particular examples of cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

### IV. Methods for Analyzing Cell Membrane Protein

In another aspect, provided herein is a method of identifying and characterizing cell membrane proteins on a cell of interest. The method utilizes a glycan-tethered stabiligase as described herein to label free N-termini of cell membrane proteins robustly and specifically with label probes on the extracellular surface of the cell of interest. The labelled cell membrane protein can further be isolated and characterized.

The method includes steps of: a) providing a cell comprising a glycan-tethered stabiligase as described herein and a population of cell membrane proteins; b) contacting the cell with a label probe comprising an ester substrate and a detectable label under conditions wherein the glycan-tethered stabiligase attaches the label probe to the population of cell membrane proteins to form a population labelled cell membrane proteins; c) isolating the labelled cell membrane proteins; and d) analyzing the labelled cell membrane proteins.

Suitable label probes for practice with the subject method includes a peptide ester substrate and a detectable label.

The term “peptide ester substrate” used in the context of the label probe refers generally to any peptide ester or peptide thioester having a chemical moiety that is capable of being utilized during the enzymatic action of the subject stabiligase that results in the specific labeling of the N-termini of proteins (e.g., cell membrane proteins) by the stabiligase. The term “peptide ester” refers generally to any peptide in which one carboxyl group of the peptide is esterified, i.e., is of the structure —CO—O—R. In some embodiments, a peptide ester can serve as a substrate for the subject stabiligase such that the peptide is added to the α-amino group of polypeptides to form the structure —CO—NH—R, thus labeling the polypeptide. The esterified carboxyl terminus of the peptide ester, which serves as a stabiligase cleavage site (i.e., the site for the nucleophilic attack by a free sulfhydryl group on stabiligase). In some embodiments, a peptide ester can carry a detectable label and a site for proteolysis or another form of chemical cleavage (e.g., through introduction of photolabile, acid-labile, or base-labile functional groups). In some embodiments, the term “peptide ester” includes any peptide thioester such as any peptide in which one carboxyl group of the peptide is thioesterified, i.e., is of the structure —CO—S—R. A useful peptide ester for use with the subject methods can be any synthetic peptide in which one carboxyl group of the peptide is esterified, i.e., is of the structure —CO—O—R, or thiesterified, i.e., is of the structure —CO—S—R, respectively. The peptide ester can serve as a substrate for a stabiligase described herein such that the peptide is added to the α-amino group of a cell membrane protein on a cell of interest to form the structure —CO—NH—R, thus labeling the cell membrane protein. A peptide ester can be synthesized using any method known to those in the art, including, but not limited to, solid phase fMOC chemistry modified for an ester bond (Braisted et al., Methods in Enzymology, 1997, 289:298-313; Jackson et al., Science, 1994, 266:243-247). The amino acid sequence of the peptide ester can contain natural amino acid residues, noncanonical amino acid residues, unnatural amino acid residues, and the like. An unnatural amino acid residue can be found at any position of the peptide sequence.

The label probe further includes a detectable label. As used herein, a “detectable label” includes a moiety that has at least one element, isotope, or functional group incorporated into the moiety which enables detection of the molecule, e.g., a protein or polypeptide, or other entity, to which the label is attached. Labels can be directly attached (i.e., via a bond) or can be attached by a tether (such as, for example, an optionally substituted alkylene: an optionally substituted alkenylene; an optionally substituted alkynylene; an optionally substituted heteroalkylene; an optionally substituted heteroalkenylene; an optionally substituted heteroalkynylene; an optionally substituted arylene: an optionally substituted heteroarylene; or an optionally substituted acylene, or any combination thereof, which can make up a tether). It will be appreciated that the label may be attached to or incorporated into a molecule, for example, a protein, polypeptide, or other entity, at any position.

Detectable labels for use with the label probes include a composition that can be detected by mass spectrometric, spectroscopic, photochemical, biochemical, immunochemical, or chemical means. For example, useful labels include radioactive isotopes (e.g., ³H, ³⁵S, ³²P ⁵¹Cr, or ¹²⁵I) stable isotopes (e.g., ¹³C, ¹⁵N, or ¹⁸O), fluorescent dyes, electron-dense reagents, enzymes (e.g., alkaline phosphatase, horseradish peroxidase, or others commonly used in an ELISA), biotin, digoxigenin, or haptens or epitopes and proteins for which antisera or monoclonal antibodies are available. In general, a tag or label as used in the context of the present invention is any entity that may be used to detect or isolate the product of the stabiligase ligation reaction. Thus, any entity that is capable of binding to another entity may be used in the practice of the subject methods, including without limitation, substrates for enzymes, epitopes for antibodies, ligands for receptors, and nucleic acids, which may interact with a second entity through means such as complementary base pair hybridization.

In general, a detectable label can fall into any one (or more) of five classes: a) a label which contains isotopic moieties, which may be radioactive or heavy isotopes, including, but not limited to, ²H, ³H, ¹³C, ¹⁴C, ¹⁵N, ¹⁸F, ³¹F, ³²F, ³⁵S, ⁶⁷Ga, ⁷⁶Br, ^{99m}Tc (Tc-99m). ^{m}In, ¹²³I, ¹²⁵I, ¹³¹I, ¹⁵³Gd, ¹⁶⁹Yb, and ¹⁸⁶Re; b) a label which contains an immune moiety, which may be antibodies or antigens, which may be bound to enzymes (e.g., such as horseradish peroxidase); c) a label which is a colored, luminescent, phosphorescent, or fluorescent moieties (e.g., such as the fluorescent label fluoresceinisothiocyanat (FITC); d) a label which has one or more photo affinity moieties; and e) a label which is a ligand for one or more known binding partners (e.g., biotin-streptavidin, FK506-PKBP). In certain embodiments, a label comprises a radioactive isotope, preferably an isotope which emits detectable particles, such as 6 particles. In certain embodiments, the label comprises a fluorescent moiety. In certain embodiments, the label is the fluorescent label fluoresceinisothiocyanat (FITC). In certain embodiments, the label comprises a ligand moiety with one or more known binding partners. In certain embodiments, the label comprises biotin. In some embodiments, a label is a fluorescent polypeptide (e.g., GFP or a derivative thereof such as enhanced GFP (EGFP)) or a luciferase (e.g., a firefly. Renilla, or Gaussia luciferase). It will be appreciated that, in certain embodiments, a label may react with a suitable substrate (e.g., a luciferin) to generate a detectable signal. Non-limiting examples of fluorescent proteins include OFP and derivatives thereof, proteins comprising chromophores that emit light of different colors such as red, yellow, and cyan fluorescent proteins, etc. Exemplary fluorescent proteins include, e.g., Sirius, Azurite, EBPP2, TagBFP, mTurquoise, ECFP, Cerulean, TagCFP, mTFP1, mUkG1, mAG1, AcGFP1, TagGFP2, EGFP, mWasabi, EmGFP, Tag YPF, EYFP, Topaz, SYFP2, Venus, Citrine, mKO, mK02, mOrange, mOrange2, TagRFP, TagRFP⋅T, mStrawberry, mRuby, mCherry, mRaspberry, mKate2, mPlum, mNeptune, T-Sapphire, mAmetrine, mKeima. See, e.g., Chalfie, M. and Kain, S R (eds.) Green fluorescent protein: properties, applications, and protocols (Methods of biochemical analysis, v. 47). Wiley-Interscience, Hoboken, N.J., 2006, and/or Chudakov, D M. et al, Physiol Rev. 90 (3): 1103-63, 2010 for discussion of GFP and numerous other fluorescent of luminescent proteins. In some embodiments, a label comprises a dark quencher, e.g., a substance that absorbs excitation energy from a fluorophore and dissipates the energy as heat. In exemplary embodiments, the detectable label is detectable using mass spectrometry. In particular embodiments, the detectable label is an aminobutyric acid (Abu) mass tag.

Any label probe with the following generic elements may be used in the practice of the methods provided herein: tag-linker-peptide sequence-esterified carboxyl terminus. The skilled artisan will recognize that the location of the detectable label within this structure may be varied without affecting the operation of the methods provided herein.

In some embodiments, the labelled cell membrane proteins are further isolated and enriched prior to analysis. In such embodiments, the label probe may further include a capture ligand that allows for capture and isolation of labelled cell membrane proteins using a capture moiety attached to a substrate. Suitable capture ligand/moiety systems include, but are not limited to: antigen and antibody or antigen-binding fragment specific therefor; biotin and avidin, streptavidin, neutravidin, or captavidin; protein A and G; a carbohydrate and a lectin; two complementary nucleotide sequences; an effector and a receptor molecule; a hormone and a hormone binding protein; an enzyme cofactor and an enzyme; an enzyme inhibitor and an enzyme; a cellulose binding domain and cellulose fibers; immobilized aminophenyl boronic acid and cis-diol bearing molecules; xyloglucan and cellulose fibers and analogues, derivatives and fragments thereof. In exemplary embodiments, the label probes include biotin and enrichment of labelled membrane proteins are carried out by capture to a substrate that includes a avidin, streptavidin, neutravidin, and/or captavidin capture moiety. Once bound to the capture moiety, the labelled cell membrane proteins may further undergo a protease digestion (e.g., a trypsin digestion) to remove internal peptides.

In embodiments wherein isolation and enrichment of labelled cell membrane proteins is performed using a capture ligand/moiety system, the label probe may further include a cleavable linker to facilitate the release of labelled cell membrane proteins after capture. A “cleavable linker” when used in the context of label probes described herein refers generally to any element contained within the peptide that can serve as a spacer and is labile to cleavage upon suitable manipulation. Accordingly, a cleavable linker may comprise any of a number of chemical entities, including amino acids, nucleic acids, or small molecules, among others. A cleavable linker may be cleaved by, for instance, chemical, enzymatic, or physical means. Non-limiting examples of cleavable linkers include protease cleavage sites and nucleic acid sequences cleaved by nucleases. Further, a nucleic acid sequence may form a cleavable linker between multiple entities in double stranded form by complementary sequence hybridization, with cleavage effected by, for instance, application of a suitable temperature increase to disrupt hybridization of complementary strands. Examples of chemical cleavage sites include the incorporation photolabile, acid-labile, or base-labile functional groups into peptides. Non-limiting examples of a cleavage moiety or cleavable linker include ENLYFQSY (SEQ ID NO:5), ENLYFQSK (SEQ ID NO:6), ENLYPQSA (SEQ ID NO:7), AAPY (SEQ ID NO:8), AAPK (SEQ ID NO:9), and AAPA (SEQ ID NO: 10). Optional protease cleavage sites that may be included in the label probes include, but are not limited to: the site for TEV protease: EXXYXQ(S/G/A) (SEQ ID NO:11), where X corresponds to any amino acid: the site for rhinovirus 3C protease: E(T/V)LFQGP (SEQ ID NO: 12); the site for enterokinase: DDDDK (SEQ ID NO:13); the site for Factor Xa: I(DE)GR (SEQ ID NO: 14); the site for thrombin: LVPR (SEQ ID NO:15); the site for furin: RXXR (SEQ ID NO:16), where X corresponds to any amino acid; and the site for granzyme B: IEPD (SEQ ID NO:17). Some examples of the many possible moieties that may be used to esterify the carboxyl terminus of the peptide are: HO—CH₂—CO—X, where X is any amino acid, in the case of glycolate esters; HO—CHCH₃—CO—X, where X is any amino acid, in the case of lactate esters: HO—R, where R is an alkyl or aryl substituent; and HS—R, where R is an alkyl or aryl substituent.

Labelled cell membrane proteins can be analyze using any suitable method. In some embodiments, the labelled cell membrane proteins are analyzed using mass spectrometry techniques. In exemplary embodiments, the labelled cell membrane proteins are analyzed using liquid chromatography-tandem mass spectrometry (LC-MS/MS).

### Examples

### Introduction

The cell surface proteome comprises approximately 3,000 proteins and is functionally critical for cellular fate and response to environmental stimuli¹. Whereas intracellular proteins may be functionally altered by hundreds of chemically different post-translational modifications (PTMs)², proteins in the extracellular space are far more limited in variety. Proteolysis is distinctly prominent among cell surface PTMs, and membrane-embedded and secreted proteases modulate many processes including cell-cell interactions, signal transduction, and cytokine secretion³. Consequently, dysregulated proteolysis is associated with many diseases. Cleavage events on healthy and abnormal cells alike create proteoforms that commonly display extracellular neo-N-termini (FIG. 1A). Characterizing cleavage events may reveal new proteoforms that can be selectively targeted for immunotherapy⁴.

Global identification of proteolysis has been greatly improved by advances in mass spectrometry (MS) methods but characterizing extracellular proteolytic modifications are challenging subjects with current techniques⁵⁻⁸. One common approach is to isolate proteins that are proteolytically-shed into the supernatant of cell cultures⁹. Despite generating data on hundreds of shedding events, this method does not precisely identify cleavage sites and is primarily limited to proteins cleaved close to or within the membrane. Another approach is to identify proteolytic products via C- or N-terminal labeling in cell lysates^{7,10-12} Labeling takes place within the whole cell lysate and the high complexity of the proteome, as well as the challenging properties of many membrane proteins-most frequently poor stability and low abundance relative to intracellular proteins-leads to incomplete coverage of extracellular proteolysis.

Previously, we developed a proteomics technology (N-terminomics) based on subtiligase, a mechanistically-engineered ligase that can specifically label N-terminal, α-amines on diverse proteins in a complex milieu (FIG. 1B)^{13,14}. The ligase, and a compilation of engineered variants, are used for diverse biotechnological applications, including peptide cyclization and protein synthesis¹⁵. For N-terminomics, subtiligase typically transfers an N-terminal specific protein label that contains a biotin handle, a TEV-protease cleavage site, and an aminobutyric acid (Abu) mass tag^{15,16}. In brief, biotinylated proteins are enriched, proteolytically-digested, and the N-terminal peptides are identified after LC-MS-MS analysis by the α-mass-tag (Abu; FIG. 1B). By labeling proteolytic neo-N-termini in whole cell lysates with subtiligase, we have extensively annotated the substrates of soluble proteases like caspases¹⁷. In attempts to identify extracellular proteolysis, however, we found that using soluble subtiligase to label intact cells or whole cell lysates yielded few cell surface N-termini. By genetically-encoding a transmembrane subtiligase in HEK293T cells, however, we found that cell surface-displayed subtiligase showed dramatically improved labeling of membrane proteins and allowed the identification of hundreds of neo-N-termini on the cell surface¹⁸.

Here, we develop an N-terminomics approach for characterizing extracellular proteolytic modifications across diverse cell types by chemical tethering of the subtiligase variant (stabiligase) to living cells. We first site-selectively modified stabiligase with an α-nucleophile that forms a covalent linkage to extracellular glycans. Then, using N-terminomics, we profiled hundreds of neo-N-termini displayed on the surface of cell types that includes primary immune cells. Collectively, we observed 1532 proteolytic modifications across structurally and functionally diverse membrane proteins. Lastly, we applied a quantitative N-terminomics approach to reveal how prominent oncogenes, kras(g12v) and her2, induce extracellular remodeling through proteolysis.

### Results

### Labeling Neo-N-Termini Based on a Chemical-Strategy for Attaching Stabiligase to Glycans on Living Cells

We envisioned a cell surface N-terminomics platform applicable across cell types and independent of genetic modifications. To achieve this, we thought to employ a chemical strategy to tether the N-terminus of the stable subtiligase variant, stabliligase,¹⁹ to extracellular glycans on intact cells (FIG. 1). First, we designed a conjugation strategy for labeling the N-terminus of stabiligase with an α-nucleophile that would readily react with extracellular glycans after cells were treated with a mild oxidant^{20,21} (FIG. 1B-C). Auto-prodomain removal generates an N-terminal alanine (A1) on mature stabiligase; to site-selectively modify the ligase, we mutated A1 to serine (A1S) to create a vicinal α-amino-alcohol. This mutation did not alter expression or purification of stabiligase. We found that a 10 minute sodium periodate oxidation of stabiligase (A1S) is sufficient to completely convert the N-terminal amino-alcohol to a glyoxyl-aldehyde (data not shown). However, this treatment also created a minor product consistent with the oxidation of the active site cysteine, C221. To protect C221, we treated stabiligase (AIS) first with Ellman's reagent (5,5′-dithiobis-(2-nitrobenzoic acid; DTNB) to generate a TNB-C221 adduct²². We then oxidized the ligase with periodate to generate an N-terminal aldehyde, incubated overnight with either bis-aminooxy- or bis-hydrazido-reagent in molar excess to introduce the α-nucleophile handle, and lastly removed the TNB-protecting group by adding a reducing agent (FIG. 1C). This strategy produced stabiligase quantitatively functionalized with either an N-terminal α-aminooxy- or α-hydrazido-group (FIG. 1C).

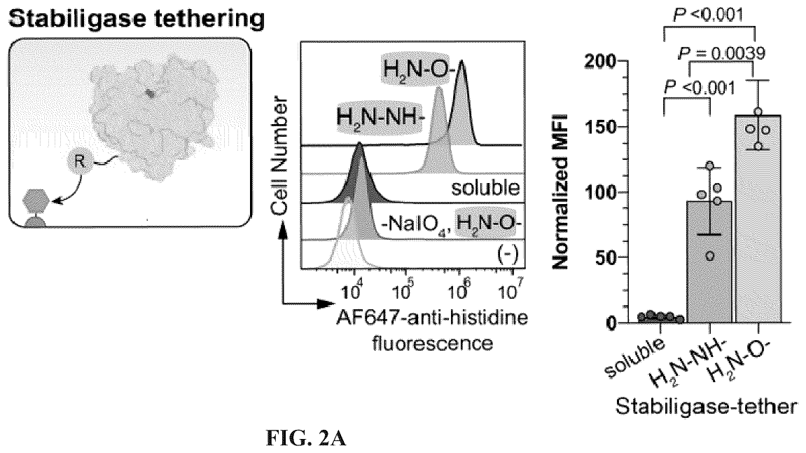

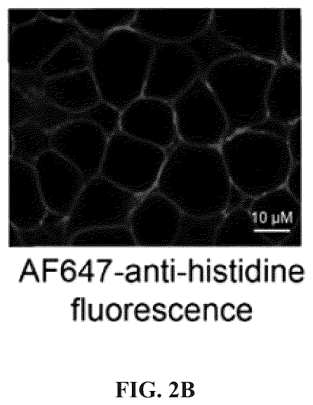

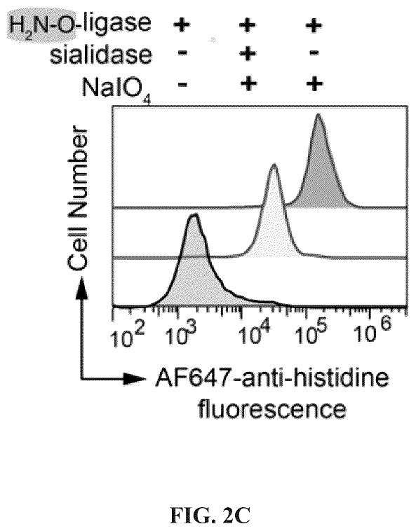

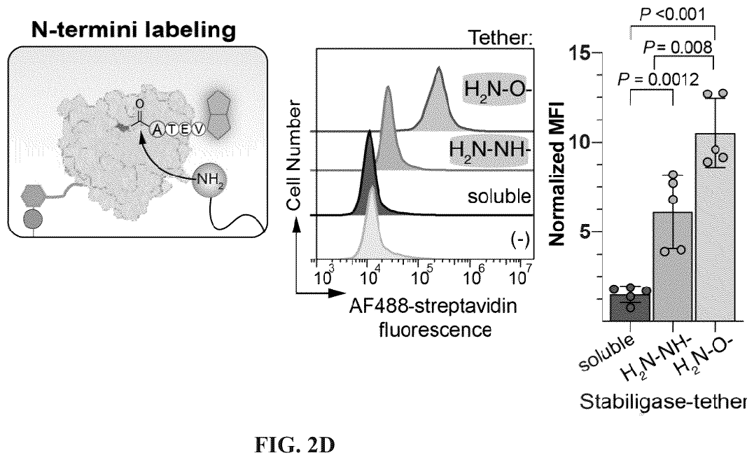

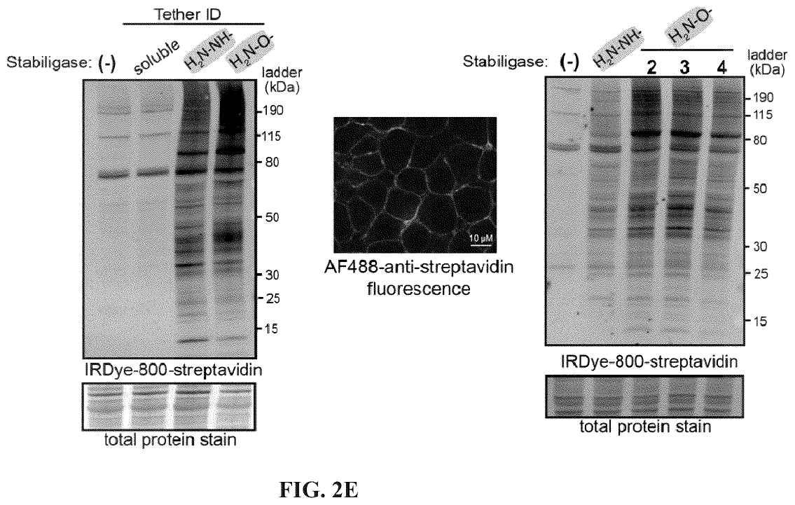

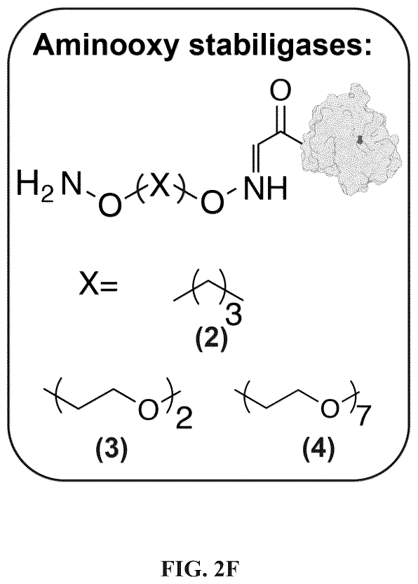

To pilot stabiligase attachment to cells, we treated HEK293T cells with sodium periodate for ten minutes on ice to form cell surface aldehydes^{20,21}, and then incubated with either of the two conjugated-stabiligases and an amine catalyst (aniline) for fifteen-minutes on ice^{14,15}. Robust tethering of both α-nucleophilic-stabiligases were determined by flow cytometry (FIG. 2A), although significantly higher levels of attachment were observed for aminooxy-stabiligase under these conditions, consistent with faster reported kinetic rates of aminooxy-nucleophiles²³. Importantly, both the α-nucleophile conjugate and periodate treatment on cells were necessary for stabiligase attachment (FIG. 2A). Furthermore, we imaged HEK293T cells stained with Alexa647-anti-histidine antibody, which monitors the C-terminal histidine tag on stabiligase, and fluorescent microscopy confirmed that the α-aminooxy-stabiligase was indeed tethered to the membrane (FIG. 2B). To assess tethering specificity, we pre-treated cells with V. cholerae sialidase^{21,24}, a hydrolase that trims the terminal sugars of glycans and observed dramatically reduced attachment of the aminooxy-stabiligase (FIG. 2C). We conclude that stabiligase derivatized with an N-terminal α-nucleophile stably attaches to cell membranes via oxidized cell surface glycans.

Alternate methods for covalent attachment of stabiligase to the cell surface were considered. We also conjugated an N-terminal alkyne onto stabiligase (A1S) to test a click-based approach. Cells were fed Ac₄GalNAz to metabolically incorporate azido-groups into cell surface glycans, and then incubated with alkynyl-stabiligase under copper-based click conditions suitable to living cells^{25,26}. However, only modest attachment of alkynyl-stabiligase was observed by flow cytometry. Given this result, we went forward an oxidative-coupling approach to tether stabiligase.

To assess the ligase activities of stabiligases tethered to the glycans of HEK293T cells, we incubated cells with a biotinylated peptide ester substrate for 15 minutes at room temperature. Flow cytometry analysis showed that biotinylation was significantly higher for cells tethered with α-nucleophilic stabiligases compared to cells incubated with a soluble stabiligase and the peptide ester (FIG. 2D). Cytoplasmic and membrane fractions were isolated and immunoblotted with streptavidin. Biotinylated protein labeling was observed almost exclusively in membrane fractions, and relative biotinylation intensities were congruent with flow cytometry results (FIG. 2E). Likewise, fluorescent microscopy of HEK293T cells stained with Alexa488-streptavidin further showed that N-terminal labeling took place along the cell membrane (FIG. 2E). Cell toxicity was evaluated after stabiligase-ligation, and we observed only a modest decrease in cell viability (15%). Collectively, these data show that the glycan-tethering (GT)-stabiligase labels the cell membrane proteome, and that α-aminooxy-functionalized stabiligase is a better conjugate for protein ligation. We were also curious to whether the proximity of the stabiligase domain to the glycan affected ligation and prepared two additional GT-stabiligases with an N-terminal aminooxy group attached via a two or seven unit poly (ethyleneglycol) (PEG) linker. Although these alternative conjugates add flexibility and theoretical distance between the glycan and ligase domain (upwards of three times the initial propanyl-linker), we observed virtually no difference in protein ligation (FIG. 2F). These data indicate that there is sufficient length, flexibility and mobility in the membrane for GT-stabiligase to access N-termini and here to after we use the original α-aminooxy-stabiligase for cell surface N-terminomics.

Mapping Neo-N-Termini with Glycan Tethered-Stabiligase N-Terminomics

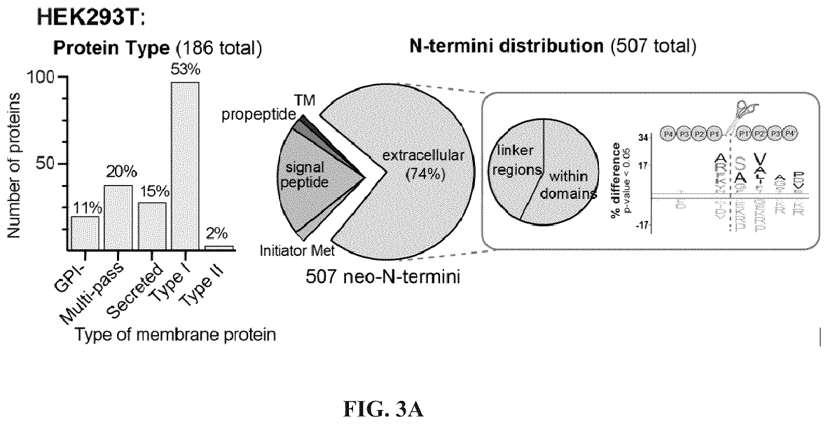

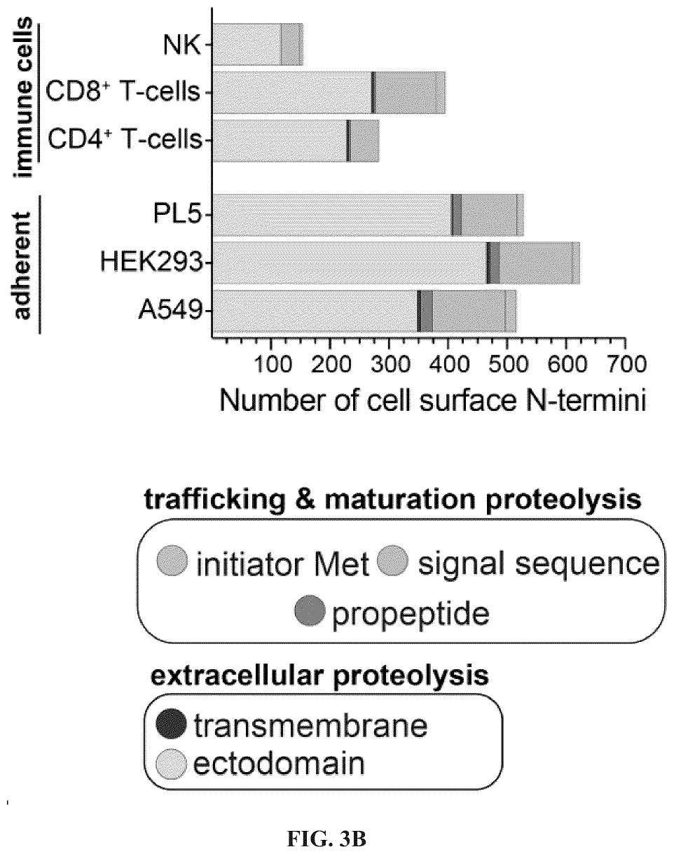

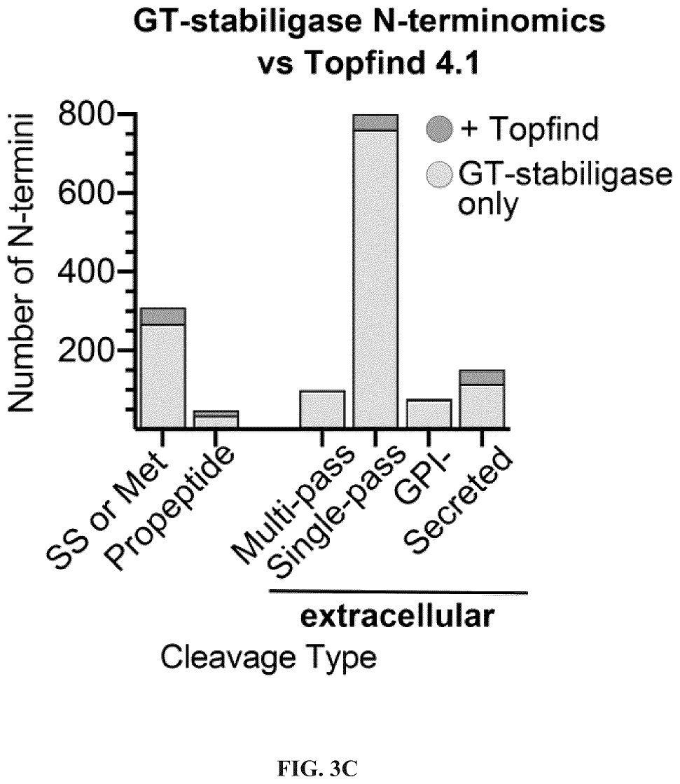

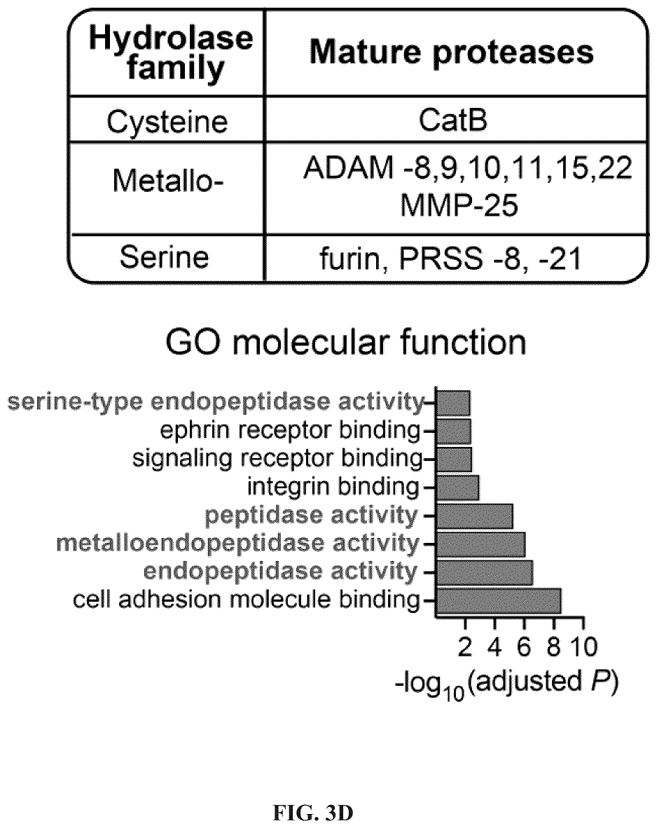

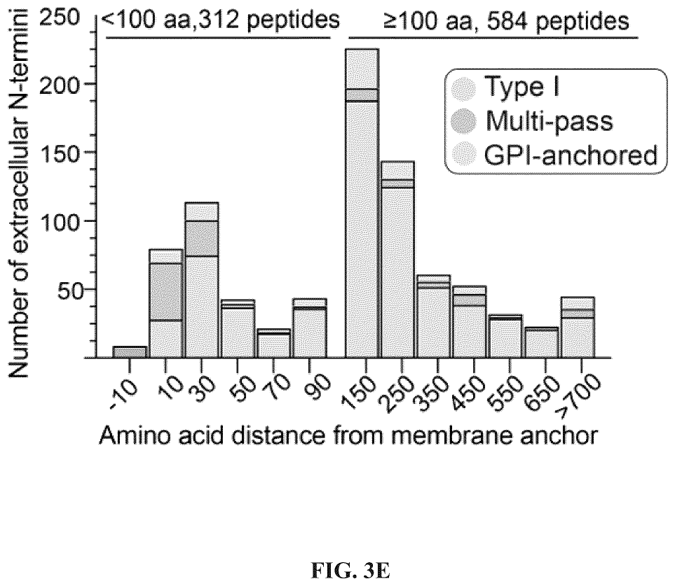

Robust GT-stabiligase tethering and subsequent biotinylation of membrane proteins on HEK293T cells encouraged us to pursue N-terminomics experiments. We treated HEK293T cells were treated with sodium periodate, GT-stabiligase, and the biotinylated peptide ester as described above. Labeled proteins were enriched using neutravidin, digested on-bead with trypsin, and lastly incubated with TEV-protease to release the mass-tagged (Abu)N-terminal peptides for LC-MS-MS analysis (FIG. 1A). Using features retrieved from UniProt knowledge database²⁷, we identified 507 Abu-tagged peptides classified as cell surface N-termini that mapped to extracellular topology of membrane proteins, extracellular secreted proteins, or GPI-anchored proteins localized in the plasma membrane (FIG. 3A). Among the proteins observed via N-terminal peptides, most proteins were type 1 single-pass proteins (67%), which is not surprising since type 1 membrane proteins comprise the majority of cell surface proteins and display an extracellular N-terminal end available to both native extracellular proteases and GT-stabiligase¹. We also identified neo-N-termini corresponding to multi-pass proteins (18%), secreted proteins (13%), and GPI-anchored (10%) proteins. In contrast, only a few cleavages were observed in type II membrane proteins (1%) which are oriented with a cytoplasmic N-terminus. We repeated the experiment using α-aminooxy-PEG₇-stabiligase and observed similar numbers of cell surface peptides (407 N-termini) which further supports the notion that GT-stabiligase is flexibly incorporated into the cell surface proteome.

Further analysis showed that identified neo-N-termini were distributed across several types of proteolysis: the removal of initiator methionine, signal peptide cleavage, propeptide removal, and post-maturation cleavage within the extracellular regions. The majority of neo-N-termini (71%) mapped to the latter group and represent potential cleavage sites of extracellular proteases. Alignment of residues (P4-P4′) flanking these inferred cleavage sites did not reveal a significant consensus sequence around the scissile bond (FIG. 3A), which suggests that multiple proteases are responsible for generating these neo-N-termini. We also considered the protein structure at extracellular cleavage sites, and mapped neo-N-termini predominantly to either interdomain, disordered regions or beta-strand regions within domains, consistent with proteolytic substrate preferences²⁸.

To evaluate utility of GT-stabiligase N-terminomics in other cell types, we applied this technology to six different cell types including adherent cells and primary immune cells (FIG. 3B). Across cell types, we observed hundreds of neo-N-termini with cleavages ranging from 500-600 for adherent cell lines and 200-400 on N-termini for immune cells. As seen with HEK293T cells, the majority of neo-N-termini localized to extracellular regions (mean, 74%) while the remainder distributed among other proteolytic maturation events, predominantly signal sequence cleavage. In total, 1532 cell surface N-termini from 449 cell surface proteins were captured across the six cell lines (FIG. 3B). Gene Ontology (GO) analysis of the proteins with proteolytic extracellular N-termini for each cell type were consistent with specific cellular processes anticipated for adherent cells and immune cells²⁹. Notably, multiple closely spaced cleavage sites were observed within some proteins. To better understand how many functionally unique cleavages were observed within proteins, we grouped closely spaced cleavages (less than three residues apart) and quantified 936 unique cleavage sites on 449 cell surface proteins.

We also assessed how GT-stabiligase N-terminomics compares to other proteomics methods. Topfind 4.1 is a database that comprises experimentally-observed N-termini from other proteomic methods (e.g., subtiligase lysate labeling,¹³ N-TAILs,⁷ COFRADIC^{6,10})³⁰ and we cross-compared Topfind N-termini to our GT-stabiligase data, grouping N-termini by cleavage type, and subdividing extracellular peptides by the type of membrane protein. Strikingly, only 143 N-termini in our data were also found in the Topfind 4.1 database (˜9%). Nearly half of these shared peptides were identified within extracellular regions on single-pass or secreted proteins, and no cleavage sites on multi-pass proteins were identified within Topfind 4.1. We also compared our data to the CSPA (Cell Surface Protein Atlas) database, which used cell surface capture (CSC) proteomics to identify 1492 cell surface proteins across 41 human cell types^{20,31}. As to be expected, we observed significant overlap in proteins between GT-stabiligase N-terminomics and CSPA (67%). Notably, proteins uniquely identified by GT-stabiligase were modestly glycosylated (median, 2 glycosites). We speculate that these proteins were not identified in CSPA because CSC proteomics requires glycosylation for enrichment whereas surface-anchored GT-stabiligase may label neighboring proteins. These comparisons further support the notion that GT-stabiligase yields broad coverage of N-termini on the cell surface with distinct utility relative to other methods.

N-terminomics with GT-stabiligase also gives several lines of evidence as to which proteases are present and active on the cell surface. Proteases are commonly synthesized as inactive precursors that require the removal of an inhibitory N-terminal propeptide for activation³². We observed 57 neo-N-termini localized to the pro-mature junction of proteins significantly enriched in endopeptidase activity as determined by molecular function analysis by gene ontology (GO) analysis (FIG. 3D)²⁹. In total, we observed 11 mature, extracellular proteases from several hydrolase families, including seven metalloproteases. The latter group includes 4 catalytically-active ADAMs, dedicated sheddases that cleave proteins within their juxtamembrane region⁹, and we thought that their activity should be reflected in the N-terminomics data. To estimate how many shed proteins were observed, we approximated the physical distance of the cleavage sites to the membrane by the amino acid distances. About 140 cleavage sites were located within 30 amino acids of the membrane and are considered candidate shed proteins (FIG. 3E). Consistent with this hypothesis, we observed well-studied examples of shed proteins including Notch (e.g., Notch 1,2)³³, receptor kinases (e.g., PTK7, PTPRK)³⁴, syndecans (e.g., SDC-1,-4)³⁵, and cell surface receptors (e.g., CD99, CD44, CCR6)⁹. Finally, we also analyzed the secondary structure, relative domain distances, and solvent accessibility as resources to gain additional insight into extracellular cleavage events. For single-pass membrane proteins, which accounts for the majority of proteins identified, over half of neo-N-termini (65%) were located between the first and last extracellular domain. Although these events do not likely represent entire extracellular shedding, the position of these N-termini suggest significant functional impact. Of note, we also observed intra-domain cleavages and these include well-studied autoproteolytic domains such as the SEA domain of the serine protease, matriptase, and the GPS domains of adhesion g-protein coupled receptors AGR2 and ADGR6³⁶. These N-termini precisely match previously annotated proteolytic sites^{27,37}. Together, these findings validate that GT-stabiligase N-terminomics is a useful technology to capturing proteolysis in a broad manner and that precise N-termini positioning provides utility for interpreting cleavage events.

### Profiling Proteolytic Changes to the Cell Surface Landscape Induced by Oncogenes

Cellular disease states are commonly associated with dysregulated proteolytic modifications, but identifying and quantifying these cleavages induced by specific oncogenes remains challenging. We previously quantified oncogene-induced changes in the surface expression of membrane proteins using an immortalized, non-tumorigenic cell line (MCF10A) transformed with individual oncogenes^{38,39}. Two oncogenes, kras^{G12V} and her2, contributed to significant alterations to the cell surface proteome through changes in both protein expression and glycosylation, and we wondered if these transformations might also alter the proteolytic landscape. Importantly, we previously found that CSC proteomics was not biased by glycan alterations³⁸. Using flow cytometry, we first assessed whether glycan variations may affect the tethering of GT-stabiligase or ligation. Encouragingly, no significant differences were observed among the parent MCF10A transduced with an empty vector (ev) and the two oncogenic cell lines (data not shown).

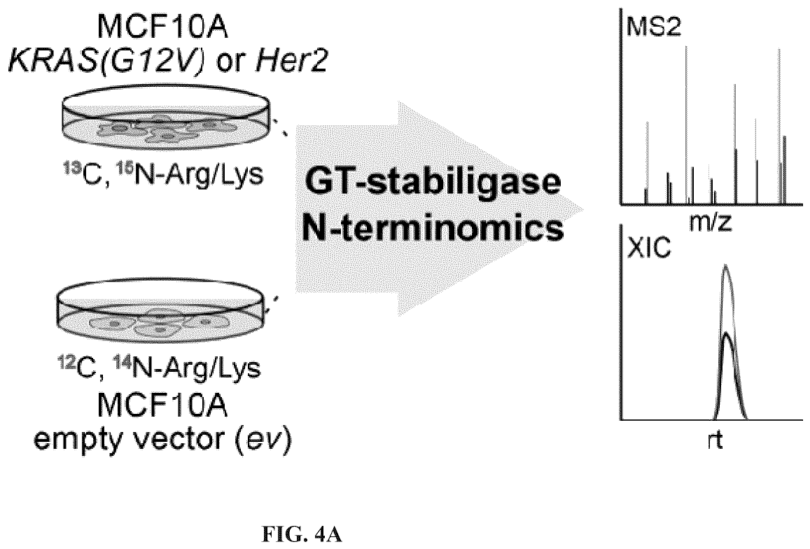

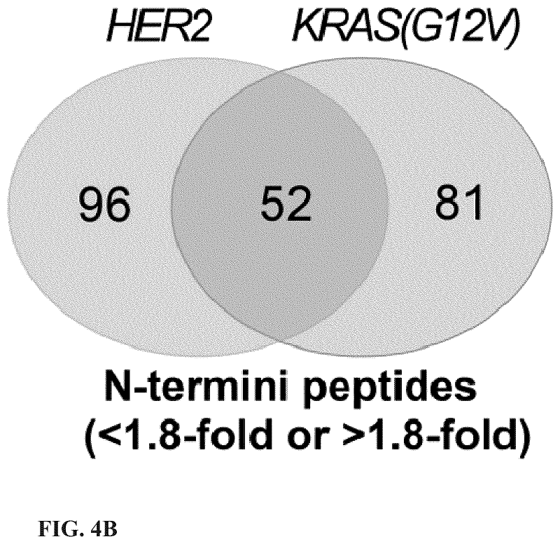

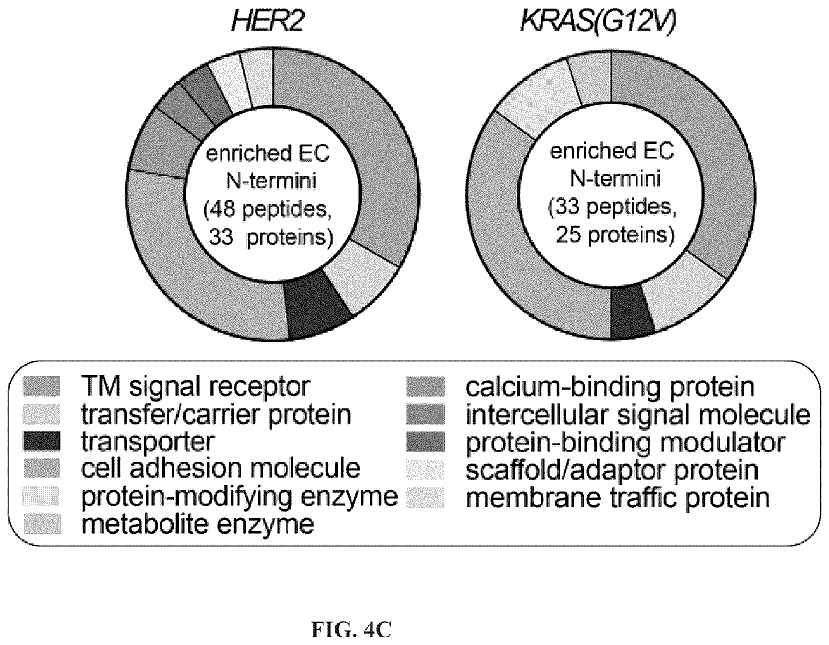

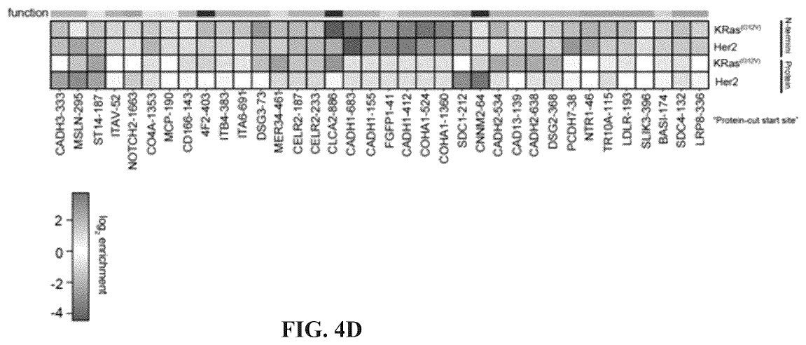

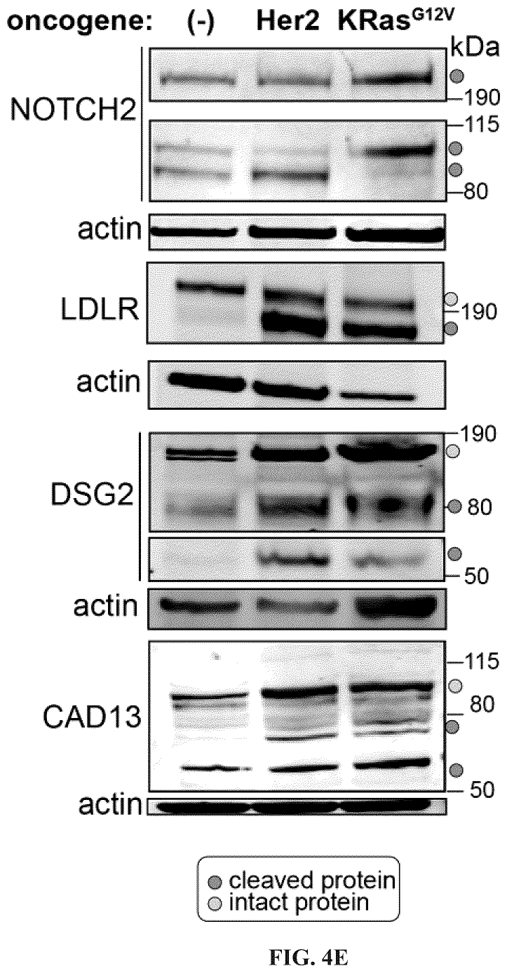

For quantitative N-terminomics, MCF10A cell lines were cultured in stable isotopic labeling of amino acids (SILAC) media. The oncogene-transformed (her2 or kras(g12v)) cell lines were combined with parental MCF10A cells transformed with an empty vector (ev), labeled with GT-stabiligase, and incubated with the peptide ester as described above (FIG. 4A). N-terminomics was performed on five biological replicates for both oncogene sets. From these we quantified 303 neo-N-terminal peptides mapped to 151 proteins, and observed 233 N-termini on 89 proteins with differential abundances (1.8-fold threshold). Among these N-termini, 35-40% of extracellular neo-N-termini overlapped between the Her2-overexpression and KRas^{G12V} datasets, and the fold-change trends were similar for the vast majority of N-termini (FIG. 4B). In both oncogenic-transformations, as shown in FIG. 4C, enriched extracellular neo-N-termini predominantly localized to cell adhesion and transmembrane signal receptors, two pathways known to undergo proteolytic modifications^{34,40}.

Next, we assessed whether changes in cell surface N-termini coincided with differences in protein abundance in the presence of either oncogene. We plotted the ratios of extracellular neo-N-termini alongside protein abundance values, as previously determined by CSC proteomics (FIG. 4D). As shown in FIG. 4D, 42 neo-N-termini with greater than 1.8-fold change in abundance mapped to 31 proteins. 80% of these proteins were observed by CSC, and interestingly, the protein abundance values were modestly correlated with N-termini abundance. We note that proteolytic removal of large extracellular domains may contribute to contradictory changes. For instance, syndecan-4 (SDC4) shedding is highly upregulated in both oncogene datasets and the protein was not observed in CSC proteomics. It is likely that cleavage leaves behind a juxtamembrane, neo-N-terminus not suitable for CSC enrichment. Transcript levels for SDC4, however, are not significantly altered in the presence of kras(g12v). Similar observations were made with individual oncogene datasets (data not shown). Together these data indicates that while oncogenes induce proteolytic alterations to the cell surface landscape through factors in addition to modifying the abundance levels of proteolytic substrates.

To provide additional validation, we selected four proteins (Notch2, DSG-2, LDLR, and T-cadherin) for whom commercially available antibodies recognize both the full-length or cleaved proteoforms for immunoblot analysis (FIG. 4E). The signaling receptor Notch2 is activated by a series of proteolytic cleavages. Mature Notch2 is cleaved by furin (S1 site) and ligand-binding induces the membrane-proximal shedding (S2 site)³³. In the presence of KRas^{G12V}, N-termini were observed at both sites and an increase cleavage at S1 was concurrent with decreased S2 cleavage, whereas only an enriched S2 cleavage site was observed in Her2-expressing cells. For both cleavage sites, the N-termini ratios were in good agreement with the protein intensities visualized by immunoblot analysis. Proteolysis of cell-adhesion protein DSG-2 plays a role in both cancer and inflammatory cells⁴¹, and the neo-N-termini characterized here map to domains reportedly cleaved by metalloproteases and ADAM-proteases⁴¹. Immunoblot analysis showed two intense bands beneath the intact DSG-2 protein in lysates of KRas^{G12V} and Her2 cells consistent neo-N-termini locations. The LDLR receptor is involved in lipid homeostasis among other functions⁴². The enriched neo-N-terminus of LDLR was observed in both oncogene datasets, and matches a previous report that a metalloprotease cleavage site that results in loss of LDL-class A ligand binding domains 1-4⁴². In agreement with these data, we observe strong protein signal for a species that matches the cleavage event. Lastly, we observed enriched N-termini mapped to propeptide and extracellular cleavages of a GPI-linked cadherin called T-cadherin (CAD13) which affects cell migration in various cancer types. Similar to our N-terminomics results, we indeed observe increased proteolytic bands consistent with its propeptide-activation and further extracellular cleavage for both Her2- and KRas^{G12V}-expressing cells. While it is not practical to test all our MS data by western blot, these examples and the fact that we find other reported cleavages precisely matching literature reports.

### REFERENCES

1 Bausch-Fluck, D. et al. The in silico human surfaceome. Proc Natl Acad Sci USA 115, E10988-E10997, doi: 10.1073/pnas. 1808790115 (2018).
2 Aebersold, R. et al. How many human proteoforms are there? Nat Chem Biol 14, 206-214, doi: 10.1038/nchembio.2576 (2018).
3 Werb, Z. ECM and cell surface proteolysis: regulating cellular ecology. Cell 91, 439-442, doi: 10.1016/s0092-8674 (00) 80429-8 (1997).
4 Dudani, J. S., Warren, A. D. & Bhatia, S. N. Harnessing Protease Activity to Improve Cancer Care. Annual Review of Cancer Biology, Vol 2 2, 353-376, doi: 10.1146/annurev-cancerbio-030617-050549 (2018).
5 Griswold, A. R. et al. A Chemical Strategy for Protease Substrate Profiling. Cell Chem Biol 26, 901-907 e906, doi: 10.1016/j.chembiol.2019.03.007 (2019).
6 Staes, A. et al. Protease Substrate Profiling by N-Terminal COFRADIC. Methods Mol Biol 1574, 51-76, doi: 10.1007/978-1-4939-6850-3_5 (2017).
7 Kleifeld, O. et al. Identifying and quantifying proteolytic events and the natural N terminome by terminal amine isotopic labeling of substrates. Nat Protoc 6, 1578-1611, doi: 10.1038/nprot.2011.382 (2011).
8 Dix, M. M., Simon, G. M. & Cravatt, B. F. Global identification of caspase substrates using PROTOMAP (protein topography and migration analysis platform). Methods Mol Biol 1133, 61-70, doi: 10.1007/978-1-4939-0357-3_3 (2014).
9 Lichtenthaler, S. F., Lemberg, M. K. & Fluhrer, R. Proteolytic ectodomain shedding of membrane proteins in mammals-hardware, concepts, and recent developments. EMBO J 37, doi: 10.15252/embj.201899456 (2018).
10 Staes, A. et al. Selecting protein N-terminal peptides by combined fractional diagonal chromatography. Nat Protoc 6, 1130-1141, doi: 10.1038/nprot.2011.355 (2011).
11 Van Damme, P. et al. Complementary positional proteomics for screening substrates of endo- and exoproteases. Nat Methods 7, 512-515, doi: 10.1038/nmeth. 1469 (2010).
12 Prudova, A. et al. TAILS N-Terminomics and Proteomics Show Protein Degradation Dominates over Proteolytic Processing by Cathepsins in Pancreatic Tumors. Cell Reports 16, 1762-1773, doi: 10.1016/j.celrep.2016.06.086 (2016).
13 Calvo, S. E. et al. Comparative Analysis of Mitochondrial N-Termini from Mouse, Human, and Yeast. Mol Cell Proteomics 16, 512-523, doi: 10.1074/mcp.M116.063818 (2017).
14 Abrahmsen, L. et al. Engineering subtilisin and its substrates for efficient ligation of peptide bonds in aqueous solution. Biochemistry 30, 4151-4159, doi: 10.1021/bi00231a007 (1991).
15 Weeks, A. M. & Wells, J. A. N-Terminal Modification of Proteins with Subtiligase Specificity Variants. Curr Protoc Chem Biol 12, e79, doi: 10.1002/cpch.79 (2020).
16 Julien, O. et al. Quantitative MS-based enzymology of caspases reveals distinct protein substrate specificities, hierarchies, and cellular roles. Proc Natl Acad Sci USA 113, E2001-2010, doi: 10.1073/pnas. 1524900113 (2016).
17 Agard, N. J., Maltby, D. & Wells, J. A. Inflammatory Stimuli Regulate Caspase Substrate Profiles. Molecular Proteomics & Cellular 9, 880-893, doi: 10.1074/mcp.M900528-MCP200 (2010).
18 Weeks, A. M., Byrnes, J. R., Lui, I. & Wells, J. A. Mapping proteolytic neo-N termini at the surface of living cells. Proc Natl Acad Sci USA 118, doi: 10.1073/pnas.2018809118 (2021).
19 Weeks, A. M. & Wells, J. A. Engineering peptide ligase specificity by proteomic identification of ligation sites. Nat Chem Biol 14, 50-57, doi: 10.1038/nchembio.2521 (2018).
20 Wollscheid, B. et al. Mass-spectrometric identification and relative quantification of N-linked cell surface glycoproteins. Nat Biotechnol 27, 378-386, doi: 10.1038/nbt.1532 (2009).
21 Zeng, Y., Ramya, T. N., Dirksen, A., Dawson, P. E. & Paulson, J. C. High-efficiency labeling of sialylated glycoproteins on living cells. Nat Methods 6, 207-209, doi: 10.1038/nmeth. 1305 (2009).
22 Masamune, S. et al. Bio-Claisen Condensation Catalyzed by Thiolase from Zoogloea—Ramigera—Active-Site Cysteine Residues. J Am Chem Soc 111, 1879-1881, doi: DOI 10.1021/ja00187a053 (1989).
23 Wang, S. J. et al. Saline Accelerates Oxime Reaction with Aldehyde and Keto Substrates at Physiological pH. Scientific Reports 8, doi: ARTN 2193 10.1038/s41598-018-20735-0 (2018).
24 Debets, M. F. et al. Metabolic precision labeling enables selective probing of O-linked N-acetylgalactosamine glycosylation. Proc Natl Acad Sci USA 117, 25293-25301, doi: 10.1073/pnas.2007297117 (2020).
25 Mockl, L. et al. Quantitative Super-Resolution Microscopy of the Mammalian Glycocalyx. Dev Cell 50, 57-72 e56, doi: 10.1016/j.devcel.2019.04.035 (2019).
26 Hong, V., Steinmetz, N. F., Manchester, M. & Finn, M. G. Labeling live cells by copper-catalyzed alkyne-azide click chemistry. Bioconjug Chem 21, 1912-1916, doi: 10.1021/bc100272z (2010).
27 The UniProt Consortium. UniProt: the universal protein knowledgebase. Nucleic Acids Research 45, D158-D169, doi: 10.1093/nar/gkw1099 (2016).
28 Madala, P. K., Tyndall, J. D. A., Nall, T. & Fairlie, D. P. Update 1 of: Proteases Universally Recognize Beta Strands In Their Active Sites. Chemical Reviews 110, Pr1-Pr31, doi: 10.1021/cr900368a (2010).
29 Liao, Y., Wang, J., Jaehnig, E. J., Shi, Z. & Zhang, B. WebGestalt 2019: gene set analysis toolkit with revamped UIs and APIs. Nucleic Acids Res 47, W199-W205, doi: 10.1093/nar/gkz401 (2019).
30 Fortelny, N., Yang, S., Pavlidis, P., Lange, P. F. & Overall, C. M. Proteome TopFIND 3.0 with TopFINDer and PathFINDer: database and analysis tools for the association of protein termini to pre- and post-translational events. Nucleic Acids Res 43, D290-297, doi: 10.1093/nar/gku1012 (2015).
31 Bausch-Fluck, D. et al. A mass spectrometric-derived cell surface protein atlas. Plos One 10, e0121314, doi: 10.1371/journal.pone.0121314 (2015).
32 Boon, L., Ugarte-Berzal, E., Vandooren, J. & Opdenakker, G. Protease propeptide structures, mechanisms of activation, and functions. Crit Rev Biochem Mol Biol 55, 111-165, doi: 10.1080/10409238.2020.1742090 (2020).
33 Kopan, R. & Ilagan, M. X. The canonical Notch signaling pathway: unfolding the activation mechanism. Cell 137, 216-233, doi: 10.1016/j.cell.2009.03.045 (2009).
34 Huang, H. Proteolytic Cleavage of Receptor Tyrosine Kinases. Biomolecules 11, doi: 10.3390/biom11050660 (2021).
35 Fitzgerald, M. L., Wang, Z. H., Park, P. W., Murphy, G. & Bernfield, M. Shedding of syndecan-1 and -4 ectodomains is regulated by multiple signaling pathways and mediated by a TIMP-3-sensitive metalloproteinase. J Cell Biol 148, 811-824, doi: DOI 10.1083/jcb.148.4.811 (2000).
36 Macao, B., Johansson, D. G., Hansson, G. C. & Hard, T. Autoproteolysis coupled to protein folding in the SEA domain of the membrane-bound MUC1 mucin. Nat Struct Mol Biol 13, 71-76, doi: 10.1038/nsmb1035 (2006).
37 Tseng, C. C. et al. Matriptase shedding is closely coupled with matriptase zymogen activation and requires de novo proteolytic cleavage likely involving its own activity. Plos One 12, doi: ARTN e0183507 10.1371/journal.pone.0183507 (2017).
38 Leung, K. K. et al. Broad and thematic remodeling of the surfaceome and glycoproteome on isogenic cells transformed with driving proliferative oncogenes. Proc Natl Acad Sci USA 117, 7764-7775, doi: 10.1073/pnas. 1917947117 (2020).
39 Martinko, A. J. et al. Targeting RAS-driven human cancer cells with antibodies to 39 upregulated and essential cell-surface proteins. Elife 7, doi: 10.7554/eLife.31098 (2018).
40 Berx, G. & van Roy, F. Involvement of Members of the Cadherin Superfamily in Cancer. Csh Perspect Biol 1, doi: ARTN a003129 10.1101/cshperspect.a003129 (2009).
41 Kamekura, R. et al. Inflammation-induced desmoglein-2 ectodomain shedding compromises the mucosal barrier. Mol Biol Cell 26, 3165-3177, doi: 10.1091/mbc.E15-03-0147 (2015).
42 Banerjee, S. et al. Proteolysis of the low density lipoprotein receptor by bone morphogenetic protein-1 regulates cellular cholesterol uptake. Sci Rep 9, 11416, doi: 10.1038/841598-019-47814-0 (2019).
43 Lim, S. A. et al. Targeting a proteolytic neoepitope on CUB domain containing protein 1 (CDCP1) for RAS-driven cancers. J Clin Invest 132, doi: 10.1172/JCI154604 (2022).

A

1

-A

8

. (canceled)

B1-B3. (canceled)

C1-C7. (canceled)

1. A cell comprising a stabiligase tethered to extracellular glycans on intact human cells.

2. The cell of claim 1, wherein the stabiligase is tethered to a glycan on the cell membrane protein by an oxime or a hydrazone bond.

3. The cell of claim 1, wherein the cell is a mammalian cell.

4. The cell of claim 1, wherein the cell is a cancer cell.

5. A method of making a cell comprising a membrane-tethered stabiligase, comprising oxidative coupling a stabiligase to a cell-membrane protein on the surface of the cell.

6. The method of claim 5, wherein the method comprises:
a) providing a cell comprising a cell membrane protein with an aldehyde group;
b) contacting the cell with a stabiligase comprising a nucleophilic group under conditions wherein the aldehyde group of the cell membrane protein and the nucleophilic group form a bond, thereby tethering the stabiligase to the cell membrane protein of the cell.

7. The method of claim 6, wherein the cell membrane protein comprises a glycan comprising the aldehyde group.

8. The method of claim 6, wherein the nucleophilic group is an α-aminooxy- or α-hydrazido-group.

9. A stabiligase comprising an N-terminal an α-aminooxy- or α-hydrazido-group.

10. The stabiligase of claim 9, wherein the stabiligase is a variant of a stabiligase having the amino acid sequence of any one of SEQ ID NOs: 1-4.

11. The stabiligase of claim 9, wherein the stabiligase comprises at least 1, 2, 3, 4, 5, 6, 7, 8, 9, 10, 11, 12, 13, 14, 15, 16, 17, 18, 19 or 20 amino acid modifications as compared to the stabiligase of SEQ ID NO:1-4.

12. A method of identifying a population of cell membrane proteins on a cell, the method comprising:
a) providing a cell comprising a membrane-tethered stabiligase and a population of cell membrane proteins;
b) contacting the cell with a label probe comprising an ester substrate and a detectable label under conditions wherein the membrane-tethered stabiligase attaches the label probe to the population of cell membrane proteins to form a population labelled cell membrane proteins;
c) isolating the labelled cell membrane proteins; and
d) analyzing the isolated labelled cell membrane proteins; therein identifying the population of cell membrane proteins on the cell.

13. The method of claim 12, wherein the label probe comprises a capture ligand and the isolating step c) comprises capturing the labelled cell membrane proteins using a substrate comprising a capture moiety.

14. The method of claim 12, wherein the capture ligand is biotin and the capture moiety is selected from the group consisting of: avidin, streptavidin, neutravidin, and captavidin.

15. The method of claim 12, wherein the detectable label is detectable by mass spectrometry.

16. The method of claim 15, wherein the detectable label is an aminobutyric acid (Abu) tag.

17. The method of claim 12, wherein cell membrane proteins are analyzed in step d) using liquid chromatography with tandem mass spectrometry (LC-MS/MS).

18. The method of claim 12, wherein the cell is a cancer cell.
